(19) United States
(12) Patent Application Publication
(10) Pub. No.: US 2026/0210842 A1
(43) Pub. Date: Jul. 23, 2026

(54) SYSTEM FOR REAL-TIME MEASUREMENT OF PHOTOCHEMICAL PRODUCTION SYSTEMS

(71) Applicant: Colorado State University Research Foundation, Fort Collins, CO (US)

(72) Inventor: John C. Mizia, Fort Collins, CO (US)

(21) Appl. No.: 19/168,443

(22) PCT Filed: Mar. 22, 2024
(86) PCT No.: PCT/US2024/021156

§ 371 Date: Sep. 24, 2025

Related U.S. Application Data
(60) Provisional application No. 63/454,404, filed on Mar. 24, 2023.

(51) Int. Cl. G01N 21/27; G01N 21/77; G01N 21/78; G01N 33/569

(52) U.S. Cl. CPC G01N 21/272; G01N 21/78; G01N 33/56911; G01N 33/56983; G01N 2021/7763

(57) ABSTRACT

The present invention is directed to novel systems, methods, and apparatus for the calibration, detection, and real-time dynamic measurement of in vitro photochemical reactions and associated light energy.

### CROSS-REFERENCE TO RELATED APPLICATIONS

This International PCT application claims the benefit of U.S. Provisional Application Ser. No. 63/454,404, filed Mar. 24, 2023, the specification, claims and drawings of which are incorporated herein by reference in their entirety.

### TECHNICAL FIELD

The present invention is directed to the field of light detection, in particular light detection in photochemical reactors, specifically photochemical reactors adapted for the inactivation of pathogens.

### BACKGROUND

Vaccination remains the most effective countermeasure for mitigating pandemics and has proven effective against viral and bacterial pathogens. Traditional vaccine production methods have included the use of RNA and DNA vaccines, subunit vaccines, attenuated vaccines, as well as vectored vaccines utilizing virus-like particles (VLP), adenovirus or bacterial host constructs. Inactivated vaccines have been a mainstay of vaccinology for decades. Even today, examples of inactivated vaccines include constructs for influenza, cholera, bubonic plague and polio. Inactivated viral vaccines are typically made by exposing virulent virus to chemical or physical agents, for example, formalin or b-propiolactone, in order to destroy infectivity. However, exposure to such harsh chemicals and/or physical agents may destroy viral epitopes, thus reducing or even destroying immunogenicity.

To overcome these limitations, the use of photochemical inactivation has been proposed, for example by Goodrich et al., in PCT/US2021/023996, incorporated herein by reference. Using the photoreactor of Goodrich, viral and bacterial pathogens can be inactivated through the administration of a dose of UV light in the presence of riboflavin. This photochemical reaction alters the nucleic acids present in the pathogen, specifically through the oxidation of guanine bases in the nucleic acid while preserving the pathogen's ability to replicate and preserving the potency and integrity of any antigenic proteins.

However, this process is dependent upon the irradiance of the light to drive the photochemical inactivation, and as such calibration of the light intensity used in such photoreactors is critical to pathogen inactivation. Traditional photoreactors use high-cost UV spectrometers to measure light intensity and perform necessary calibrations of the same. However, this method is costly, and difficult to maintain and cannot provide real-time measurements of an operating photobioreactor.

As such, there exists a long-felt need for a simple, cost-effective system to detect, measure, and calibrate light intensity within photobioreactor systems. There further exists a need for real-time detection and calibration of the photochemical reactions of photoreactors used to inactivate viral or bacterial particles.

### SUMMARY OF THE INVENTION

The present invention is directed to systems, methods, and apparatus for the calibration, detection, and real-time dynamic measurement of in vitro photochemical reactions and associated light energy. In a first exemplary embodiment of the present invention, a system and method for detecting light emitted into a photoreactor may include a housing containing a fluid channel configured to transfer a fluid containing a photosensitizer and a reactant from an inlet to an outlet preferably along a longitudinal axis, and at least one light source positioned adjacent to the fluid channel, again preferably along the longitudinal axis. In another preferred embodiment, the photosensitizer is a flavin, such as for example riboflavin or a psoralen, and the reactant includes a microorganism, such as a virus or bacterium. The system may further include a light detection assembly having one or more photodiodes positioned adjacent to the light source and configured to generate an electrical signal in response to the light energy produced by the at least one light source. The system may further include a controller electrically connected to said one or more photodiodes, configured to convert said electrical signal to a sensor signal that corresponds to said light energy.

In another version of the first exemplary embodiment, the system for detecting light emitted into a photoreactor the light source includes at least one inner light source adjacent the fluid channel, the at least one inner light source positioned between the fluid channel and the longitudinal axis. In other preferred aspects, the light source can include at least one outer light source adjacent the fluid channel, the fluid channel positioned between the outer light source and the longitudinal axis. In other preferred aspects, the fluid channel is a helical pathway wound about a longitudinal axis.

In another version of the first exemplary embodiment, the system for detecting light emitted into a photoreactor the light source includes one or more photodiodes positioned adjacent to the light source at one or more corresponding measurement positions along the longitudinal axis of the light source, which can further include the longitudinal axis of the inner and/or outer light sources. In other preferred aspects, the photodiode comprises a UV-A dominant diode, preferably with a detection sensitivity between 220 nm-370 nm. In another example, the light source of the system of the invention can be selected can include a fluorescent light source, an LED light source, a narrowband wavelength light source, a peak UV-B wavelength, a peak UV-C wavelength, and a peak wavelength outside of UV-B and UV-C, or any combination of the same.

In another version of the first exemplary embodiment, one or more parameters of the operation of the photoreactor can be adjusted, preferably by a controller, and more preferably automatically and/or in real-time, such that the photochemical reaction between the photosensitizer and reactant can be calibrated, optimized, or maintained in an optimal or desired state. In one example, the intensity of the light source is adjusted in response to the sensor signal. In another example, one or more light sources are activated or deactivated in response to the sensor signal. In another example, the wavelength of the light source is adjusted in response to the sensor signal. In another example, the flow rate of the fluid through the channel is adjusted in response to the sensor signal. In another example, the ratio of the photosensitizer and the microorganism is adjusted in response to the sensor signal. In still further examples, a sensor signal correspond to a fault or anomaly in the light emitted by one or more light sources.

In a second exemplary embodiment of the present invention, a system and method for measuring a photochemical reaction includes a photoreactor having a fluid channel configured to transfer a fluid from an inlet to an outlet, wherein said fluid contains a photosensitizer and a reactant. In another preferred embodiment, the photosensitizer is riboflavin, and the reactant includes a microorganism, such as a virus or bacterium.

The system may further include a fluid detection assembly positioned at the inlet and/or outlet of the fluid channel having a light source configured to direct light energy through said fluid in the fluid channel, and at least one photodiode also positioned adjacent to the fluid channel and configured to generate an electrical signal in response to the light energy produced by the light source that passes through the fluid. The system may further include a controller, electrically connected to one or more photodiodes, which is further configured to convert the electrical signal to a sensor signal that corresponds to said light energy passing through the fluid.

In another version of the first exemplary embodiment, the system for detecting light emitted into a photoreactor the light source includes at least one inner light source adjacent the fluid channel, the at least one inner light source positioned between the fluid channel and the longitudinal axis. In other preferred aspects, the light source can include at least one outer light source adjacent the fluid channel, the fluid channel positioned between the outer light source and the longitudinal axis. In other preferred aspects, the fluid channel is a helical pathway wound about a longitudinal axis.

In another version of the first exemplary embodiment, the system for detecting light emitted into a photoreactor the light source includes one or more photodiodes positioned adjacent to the light source at one or more corresponding measurement positions along the longitudinal axis of the light source, which can further include the longitudinal axis of the inner and/or outer light sources. In other preferred aspects, the photodiode comprises a UV-A dominant diode, preferably with a detection sensitivity between 220 nm-370 nm. In another example, the light source of the system of the invention can be selected can include a fluorescent light source, a fluorescent light source, an LED light source, a narrowband wavelength light source, a peak UV-B wavelength, a peak UV-C wavelength, and a peak wavelength outside of UV-B and UV-C.

In another version of the first exemplary embodiment, one or more parameters of the operation of the photoreactor can be adjusted, preferably by a controller, and more preferably automatically and/or in real-time, such that the photochemical reaction between the photosensitizer and reactant can be calibrated, optimized, or maintained in an optimal or desired state. In one example, the intensity of the light source is adjusted in response to the sensor signal. In another example, one or more light sources are activated or deactivated in response to the sensor signal. In another example, the wavelength of the light source is adjusted in response to the sensor signal. In another example, the flow rate of the fluid through the channel is adjusted in response to the sensor signal. In another example, the ratio of the photosensitizer and the microorganism is adjusted in response to the sensor signal. In a preferred embodiment, the sensor signal corresponds to a color change in the fluid. As used herein, the term “color” refers to the wavelength of visible light, also referred to as the “color spectra” and further, the steps related to detecting a change in the wavelength of light or a change in color means detecting a change in the observable color spectra resulting from a physical or chemical change occurring in the fluid that results in the change of the observable color spectra.

In another version of the first exemplary embodiment, a system for detecting emitted light is described, wherein the system can be configured to detect the wavelength of light energy applied to the system or the color spectra of the fluid. In this embodiment, a fluid channel can be configured to transfer a fluid containing at least one reactant, where at least one light source positioned adjacent to the fluid channel. Further, the system can include a light detection assembly including: one or more sensors positioned adjacent to the light source and configured to detect the light energy produced by the light source; and a controller electrically connected to said one or more sensors, configured to generate a signal that corresponds to said wavelength of the light energy or the color spectra of the fluid.

In another version of the first exemplary embodiment, a method for calibrating a photoreactor light source is described. In this embodiment, the method includes directing a fluid through a fluid channel, wherein said fluid includes a least one reactant, and further directing a light source adjacent the fluid channel and emitting a light into the fluid. Sensors are positioned adjacent to the light source and configured to detect the light energy produced by the light source and transmitting an electrical signal to a controller. The electrical signal is converted to a signal that corresponds to the wavelength of the light energy or the color spectra of the fluid. In this embodiment, the signal that corresponds to the wavelength of the light energy or the color spectra of the fluid can be compared to a control signal generated under negative or positive control parameters.

In another version of the first exemplary embodiment, a method for measuring a photochemical reaction is described. In this embodiment, the method includes establishing a photoreactor having a fluid channel configured to transfer a fluid from an inlet to an outlet wherein said fluid comprises at least one reactant, and where light energy is directed through the fluid. At least one sensor is positioned adjacent to said fluid channel and generating an electrical signal that that corresponds to the wavelength of the light energy or the color spectra of the fluid, wherein the electrical signal is converted to a sensor signal that corresponds to the wavelength of the light energy or the color spectra of the fluid.

Additional aspects of the invention may become evident based on the specification and claims presented below.

### BRIEF DESCRIPTION OF THE FIGURES

The foregoing summary, as well as the following detailed description of preferred embodiments of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown. In the drawings:

FIG. 1 is a perspective view of a photoreactor according to an exemplary embodiment of the present disclosure.

FIG. 2 is a perspective view of some of the components of the photoreactor from FIG. 1.

FIG. 3 is a perspective view of a lamp subassembly from FIG. 1.

FIG. 4 is a is a top sectional view of the lamp subassembly of FIG. 3 taken along section line 4-4 in FIG. 3.

FIG. 5 is a perspective longitudinal sectional view of the photoreactor of FIG. 2 taken along section line 5-5 in FIG. 2.

FIG. 6 is a perspective view of some of the components of the photoreactor from FIG. 1.

FIG. 7 is a top view of a coil assembly according to an exemplary embodiment of the present disclosure.

FIG. 8 is a perspective longitudinal sectional view of the coil subassembly from FIG. 7 as taken along section line 8-8 in FIG. 7.

FIG. 9 is a perspective longitudinal sectional view of a component of the coil subassembly from FIG. 7 as taken along section line 8-8 in FIG. 7.

FIG. 10 is a perspective longitudinal sectional view of a component of the coil assembly from FIG. 7 as taken along section line 8-8 in FIG. 7.

FIG. 11 is a is a perspective view of a lamp subassembly according to an exemplary embodiment of the present disclosure.

FIG. 12 is a is a top sectional view of the lamp subassembly of FIG. 11.

FIG. 13 is a is a perspective view of a lamp subassembly according to an exemplary embodiment of the present disclosure.

FIG. 14 is a is a top sectional view of the lamp subassembly of FIG. 13.

FIG. 15 is a is a perspective view of a lamp subassembly according to an exemplary embodiment of the present disclosure.

FIG. 16 is a is a top sectional view of the lamp subassembly of FIG. 15.

FIG. 17 is a is a perspective view of a lamp subassembly according to an exemplary embodiment of the present disclosure.

FIG. 18 is a is a top sectional view of the lamp subassembly of FIG. 17.

FIG. 19 shows light intensity over time using a photodiode to calibrate the light intensity of a lamp with and without a warm-up phase according to an exemplary embodiment of the present disclosure.

FIG. 20A-B shows a light detection assembly according to an exemplary embodiment of the present disclosure.

FIG. 21 shows a light detection assembly having a photodiode secured to a measurement position adjacent to a lamp according to an exemplary embodiment of the present disclosure.

FIG. 22 shows a schematic of a light detection having an array of photodiodes secured to measurement positions on a photoreactor housing and adjacent to a lamp and responsive to a controller according to an exemplary embodiment of the present disclosure.

FIG. 23 shows a schematic representation of fluid detection assembly secured to a photodiode and positioned adjacent to a fluid chamber and in-line with a lamp according to an exemplary embodiment of the present disclosure.

### DETAILED DESCRIPTION OF THE INVENTION

The invention now being generally described will be more readily understood by reference to the following examples, which are included merely for the purposes of illustration of certain embodiments of the present invention. The examples are not intended to limit the invention, as one of skill in the art would recognize from the above teachings and the following examples that other techniques and methods can satisfy the claims and can be employed without departing from the scope of the claimed invention. Indeed, while this invention has been particularly shown and described with references to preferred embodiments thereof, it will be understood by those skilled in the art that various changes in form and details may be made therein without departing from the scope of the invention encompassed by the appended claims.

Described herein are compositions, devices, systems, and methods for the detection and calibration if UV irradiance by one or more photodiodes in a photoreactor configured for the inactivation of pathogens for virus production, or for the sterilization of a substance or surface and the like. As noted above, as taught by Goodrich et al., the photosensitizer riboflavin and UV light to selectively inactivate virus and other pathogens by directed damage to nucleic acids while preserving the integrity of the proteins and other viral antigens. The nature of the photosensitizer (riboflavin) may provide for low toxicity and thus easy handling, distribution, and processing under even austere conditions.

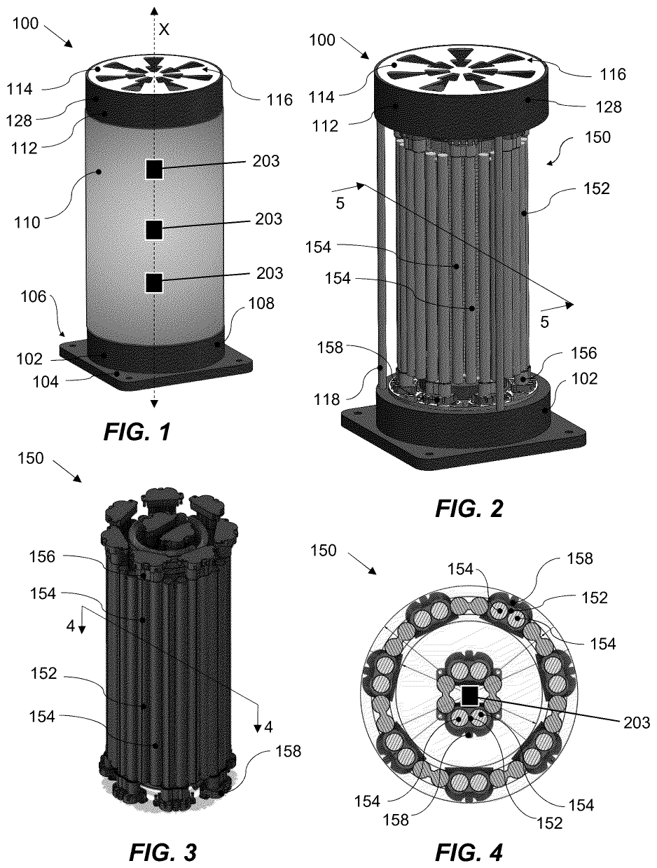

FIGS. 1-6 depict an exemplary embodiment of a photoreactor 100 for emitting and directing photons into a flowing fluid, such as a solution containing photosensitizer, such as riboflavin. The photoreactor 100 may be included in a photoreactor system which may include a pump (not shown) such as a positive displacement pump for pumping the fluid and at least one reservoir (not shown) for storing the fluid. With reference to FIG. 1, the photoreactor 100 includes a base 102 which may include a substantially flat flange portion 104 for resting on a flat surface. The flange portion 104 may define one or more apertures 106 for receiving a corresponding fastener for securing the photoreactor 100 to the flat surface. The base 102 may also include a cylindrical portion 108 extending upward from the flange portion 104 along a longitudinal axis X. The cylindrical portion 108 may include a plurality of radial apertures (not shown) for providing a passageway therethrough for electrical cables, tubing, and/or ventilation air. A reflective sleeve 110 or housing as described below may extend axially between the cylindrical portion 108 of the base 102 and a cylindrical portion 128 of a top cap 112 located at an end opposite from the base 102. The reflective sleeve 110 may have a cylindrical shape with a mirrored inner surface for reflecting light inwardly. The mirrored inner surface may comprise an oxidized coating such as ZnO2, Y2O3, ThO2, Sc2O3, MgO, Al2O3, HfO2, TiO₂, SiO2 or various combinations thereof. The top cap 112 may have a cylindrical shape with a vent plate 114 at an axially most distal end. The vent plate 114 may have or define one or more axial apertures 116 for providing a passageway therethrough for electrical cables, tubing, and/or ventilation air.

FIG. 2 shows the photoreactor 100 with the reflective shield 110 removed. One or more support rods 118 may extend axially between the cylindrical portion 108 of the base 102 and the top cap 112. The support rods 118 may support the top cap 112 by itself or in conjunction with the reflective shield 110. In some embodiments the support rods 118 may be removed and the top cap 112 may be supported only by the reflective shield 110. The cylindrical portion 108, the top cap 112, and the reflective shield 110 may house a lamp subassembly 150. The lamp subassembly 150 includes one or more lamps 152, with each lamp 152 having a base 156 for connecting one or more bulbs 154 arranged parallel to the longitudinal axis X.

As best shown in FIGS. 3 and 4, the lamp subassembly 150 has eighteen lamps 152 with each lamp 152 having two bulbs 154 (each lamp has two halves, but it is only one lamp). The base 156 may include a receptacle for receiving one or more bulbs 154 and at least two pins opposite the receptacle for making an electrical connection in a corresponding socket 158. The base 156 may be a 2G11-type base, which has four pins. It is envisioned that the lamp assembly 150 may include other lamp and/or bulb types or geometries known to those having ordinary skill in the art. The lamps 152 of the subassembly 150 may be arranged along two concentric circles and may be alternatingly mounted to the base 102 and the top cap 112. For example, as shown in FIG. 3, seven lamps 152 and sockets 158 may be arranged in a heptagon orientation extending downwardly circumscribing two inner lamps 152 that also extend downwardly. Between each pair of bulbs 154 of adjacent downwardly extending lamps 152 is a pair of upwardly extending bulbs 154. The upwardly extending lamps 152 include seven corresponding lamps 152 also arranged in the shape of a heptagon, but out of phase by 360 degrees/number of lamps 152, which is approximately 51 degrees from the seven downwardly extending lamps 152, and two corresponding inner lamps 152 that also extend upwardly, but out of phase by 90 degrees from the two downwardly extending lamps 152. Other lamp subassembly 150 arrangements have been contemplated and are discussed in more detail below.

Returning to the bulbs 154, the lamp subassembly 150 may include fluorescent bulbs configured to emit visible, ultra-violate (UV), and/or infrared light within broad or narrow bandwidths. For example, the bulbs 154 may be fluorescent bulbs configured to emit a wide bandwidth of UV-A and UV-B wavelengths, such as a bandwidth between approximately 275 nm and 375 nm. The bulbs 154 may be germicidal fluorescent bulbs configured to emit a narrow band of UV-C wavelength, such as a narrow bandwidth centered around 253.7 nm. The bulbs 154 may also include LED bulbs with narrow bandwidths, although wide bandwidths are also possible. As some non-limiting examples, the LED bulbs may be configured to have an approximately 10 nm bandwidth or smaller centered at a peak wavelength of 265 nm, 275 nm, 310 nm, 365 nm, 395 nm, or 405 nm. A peak wavelength may be the largest amplitude of a wavelength emitted from the entire spectrum of light emitted from the light source or it may be the wavelength associated with largest amount of energy emitted for a narrow bandwidth at each localized amplitude peak emitted from the light source. The lamps 152 of the lamp subassembly 150 may be configured to have any broadband or narrowband fluorescent or LED bulb or combination thereof. For example, the lamp subassembly 150 may be comprised of 18 fluorescent bulbs configured to emit wide bandwidth UV-A and UV-B wavelengths and 18 LED bulbs configured to emit 265 nm wavelength light. The lamp subassembly 150 may be configured for quick replacement of any number of the bulbs 154 as necessary without removing the entire lamp subassembly 150. In addition, as will be described in more detail below, the lamps 152 and/or the bulbs 154 may be selectively enabled, disabled or calibrated for various reasons. Moreover, some of the bulbs may be pulsed on and off to have a desired duty cycle, particularly for LED bulbs. Further, LED bulbs having different wavelengths may be cycled on and off. The following is an example of how the photoreactor 100 may be configured and reconfigured. The photoreactor 100 may have a lamp subassembly 150 comprising 36 fluorescent bulbs configured to emit wide bandwidth UV-A and UV-B wavelengths. An operator may pass a quantity of a fluid through the photoreactor 100. Next, the operator may electronically switch off 18 of the bulbs 152 for reducing power and heat generation and then pass another quantity of a fluid through the photoreactor 100. Next, the operator may reconfigure the lamp subassembly 150 to have 18 fluorescent bulbs configured to emit wide bandwidth UV-A and UV-B wavelengths and 18 LED bulbs configured to emit 265 nm wavelength light and switch back on the 18 bulbs which were previously turned off. The operator may then pass another quantity of a fluid through the photoreactor 100. Next, the operator may then turn off the 18 fluorescent bulbs and pass another quantity of a fluid through the photoreactor 100. Finally, the operator may then turn on the 18 fluorescent bulbs and turn off the 18 LED bulbs and pass another quantity of a fluid through the photoreactor 100. Although the examples above discuss fluorescent and LED bulbs configured to emit UV wavelengths of light, the lamp subassembly may comprise fluorescent and/or LED bulbs configured to emit visible or infrared wavelengths of light. In addition, the lamp subassembly 150 may also be configured to use incandescent bulbs, halogen lamps, arc lamps, and gas-discharge lamps.

In some embodiments, the UV light may have a wavelength of 170 to 400 nm, including all ranges and subranges therebetween. For example, in some embodiments, the UV light has a wavelength of 315 to 400 nm, 310 to 320 nm, 280 to 360 nm, 280 to 315 nm, or 180 to 280 nm. In some embodiments, viral particles may be treated with multiple wavelengths of light simultaneously. In some embodiments where riboflavin is used as a photosensitizer, UV light having a wavelength of 310 to 320 nm may be used. The inventors have determined that this wavelength prevents riboflavin from reacting in free solution, which results in production of undesirable oxygen free radicals. At these wavelengths, riboflavin may selectively react when intercalated with nucleic acid.

Moreover, the inventors have further determined that the light emission characteristics of UV emitting lamps 203 can vary at different points along their length resulting in inconsistent application of light energy to a fluid sample. As such, in one embodiment, the invention includes systems, methods and apparatus to calibrate the light output of one or more lamps 203 of the photoreactor 100 and further provide anomaly or fault detection that could altern the photochemical reaction of the photosensitizer and pathogen in the fluid.

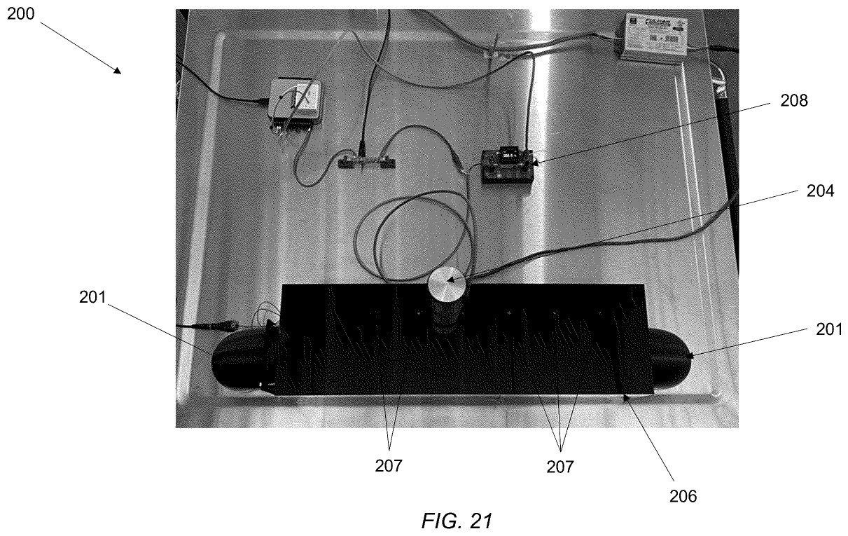

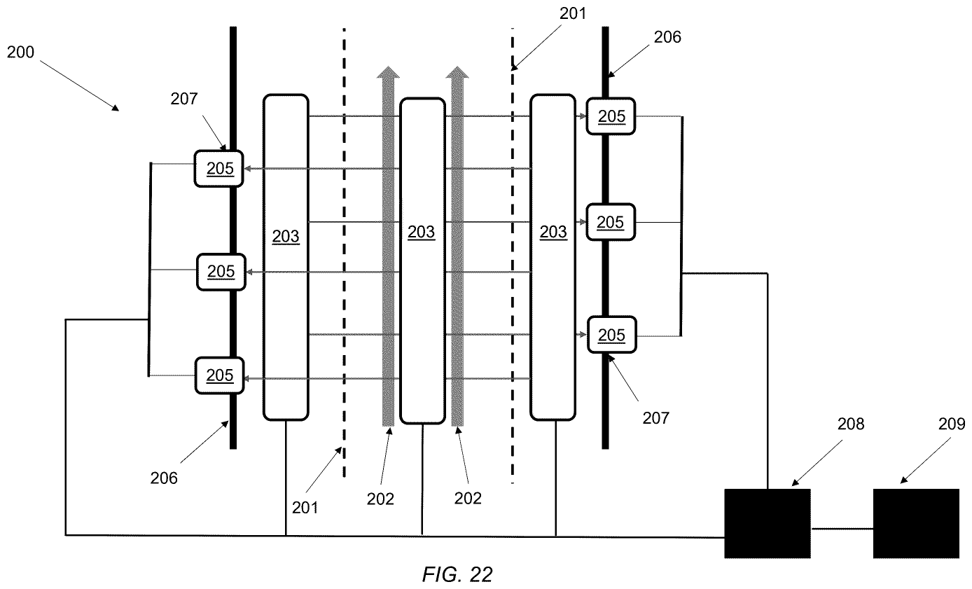

Generally referring to FIG. 22, in one embodiment the photoreactor 100 of the invention includes a light detection assembly 200 configured to detect the light intensity generated within the photoreactor 100. In this preferred embodiment, light detection assembly 200 of the invention includes one, or preferably a photodiode 205 positioned adjacent to a lamp 203 of the invention. As shown in FIG. 21, a series of photodiodes 205 positioned within a support 204 are positioned at measurement positions 207 along the length of a lamp 203, which lamp bay be adjacent to a fluid channel 201 configured to transmit a fluid 202 comprising a photosensitizer and a reactant.

In this configuration, a plurality of photodiodes 205, and preferably UV-A dominant diode with a detection sensitivity between 220 nm-370 nm, are positioned adjacent to a lamp 203 and configured to detect the intensity of its UV light energy. The photodiodes 205 can further be electrically connected to a controller 208 that is configured to detect and amplify the captured light energy into a sensor signal that can be transmitted to an output device. In this embodiment, the sensor signal corresponds to the optical emission captured by the photodiodes 205, which can be displayed as a visual or audible indication on an output device 209, such as general purpose computer or other computerized device configured to display sensor signal. An operator, or executable computer program operating on a programable computing device, can detect and analyze the sensor signals and detect anomalies or faults in the lamps 203 illumination.

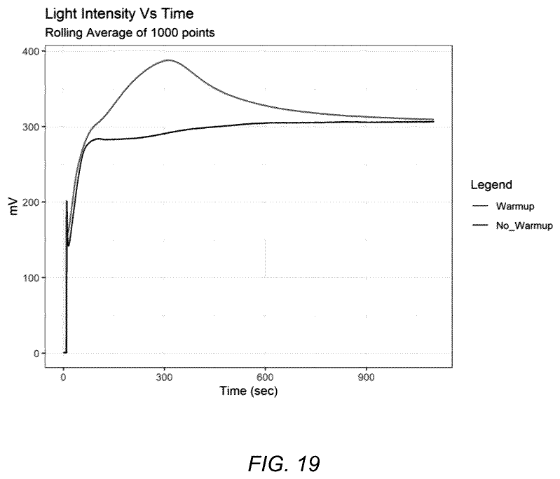

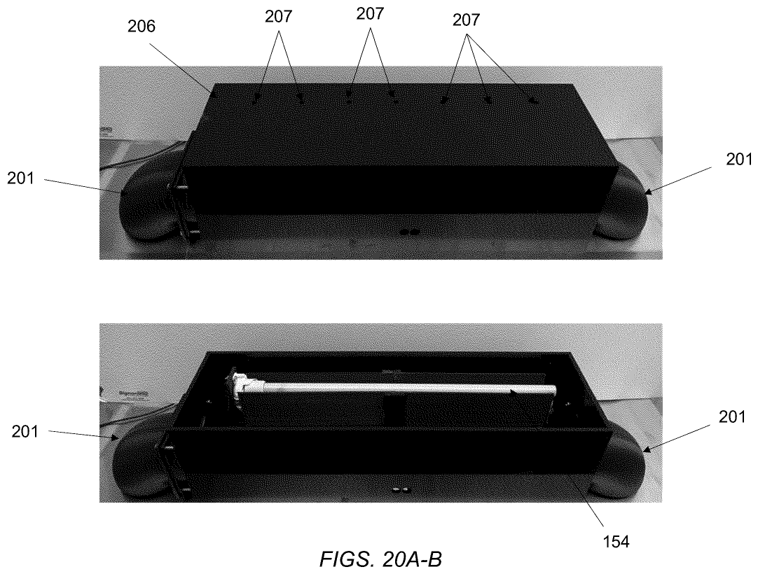

As further shown in FIG. 19, in additional embodiment, an operator or executable computer program operating on a programable computing device, generate multiple light energy measurements over time and monitor the changes in the sensor signals that correspond to light intensity within the photoreactor 100. Based on these series of sensor signals outputs, an operator can dynamically, and in real-time adjust the parameters of the photoreactor 100, such as light emission, flow rate of the fluid, the number of lamps illuminated or turned off, number of illuminated lamps, concentration of photosensitizer and/or reactant, such as a pathogen as well as wavelength of the light source.

The photodiodes 205 of the invention can be positioned on a housing 206 so as to detect the light intensity within the closed environment, of for example a photoreactor. As shown in FIGS. 1 and 22, one, or an array of photodiodes 205 can be secured to the reflective sleeve 110, or within a coil subassembly 20, for example between the inner and outer lamps of the lamp subassembly 150 as described generally herein.

The dose of the UV light may vary depending on the volume of solution being treated. For example, the dose of the UV light may be between 200-400 Joules (e.g., 300 Joules) for a volume of about 170 to 370 ml of solution. In another embodiment, the dose of the UV light may be approximately ½ Jules per milliliter of fluid passing through a fluid channel 201. As will be understood by those of skill in the art, the dosage may be adjusted up or down if the volume to be treated is above or below this range.

In some embodiments, the dose of UV light may be from about 200 Joules to about 600 Joules, for example about 200, about 225, about 250, about 275, about 300, about 325, about 350, about 375, about 400, about 425, about 450, about 475, about 500, about 525, about 550, about 575, or about 600 Joules. In some embodiments, the volume of viral preparations for illumination may be from about 200 ml to about 600 ml, for example about 200, about 225, about 250, about 275, about 300, about 325, about 350, about 375, about 400, about 425, about 450, about 475, about 500, about 525, about 550, about 575, or about 600 ml. In some embodiments, the dose of UV light may be from about 0.5 Joules/ml to about 3.0 Joules/ml. For example, the dose of UV light may be about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, or about 3.0 Joules/ml. In a preferred embodiment, the dose of the UV light may be 0.5 Joules/ml. In certain other embodiments, the UV light may include, or even exceed any of the aforementioned values by several orders of magnitude.

In some embodiments, the light treatment comprises treatment with light from a blue LED. In some embodiments, the wavelength of the light is 300 nm to 500 nm. In some embodiments, the wavelength is about 450 nm. In an embodiment, the wavelength is about 447 nm.

As discussed above, the total energy or per unit volume can be adjusted. This may be done by adjusting the pump speed, selecting a tubing 132 having a given length/diameter, and/or activating and deactivating various lamps 152 as well as changing the intensity of lamps/LED's.

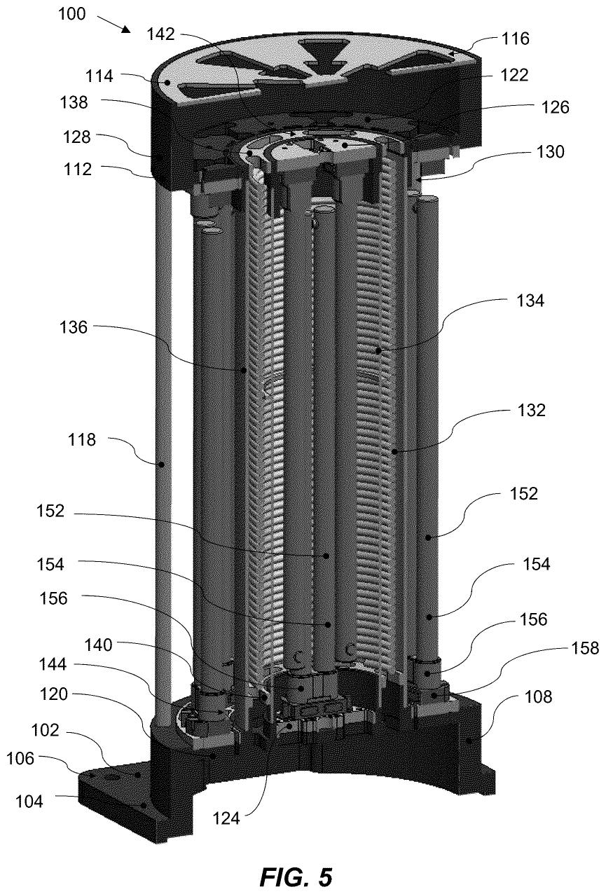

Returning to the figures, FIG. 5 is a perspective longitudinal sectional view of the photoreactor 100 showing internal features thereof. The base 102 may include an annular base mounting surface 120 extending radially inward from the cylindrical portion 108 for mounting the socket 158 of each upwardly extending lamp 152 from the outer circumferentially arranged lamps 152. Likewise, the top cap 112 may include an annular top cap mounting surface 122 extending radially inward from the cylindrical portion 128 for mounting the socket 158 of each downwardly extending lamp 152 from the outer circumferentially arranged lamps 152. Both of the base and top cap annular mounting surfaces 120, 122 may include a plurality of vent holes extending therethrough. The base 102 may also include a lower central mounting surface 124 removably attachable to the base 102 and positioned radially inwardly and centrally with respect to the base mounting surface 120. The sockets 158 of the upwardly extending inner lamps 152 may be mounted on the lower central mounting surface 124. Similarly, the top cap 112 may also include an upper central mounting surface 126 removably attachable to the top cap 112 and positioned radially inwardly and centrally with respect to the top cap mounting surface 122. The sockets 158 of the downwardly extending inner lamps 152 may be mounted on the upper central mounting surface 126.

The photoreactor 100 may also include a coil subassembly 130 positioned between the inner and outer lamps 152 of the lamp subassembly 150. The coil subassembly 130 may include an inner cylindrical shield 134 positioned adjacent and radially outward with respect to the inner lamps 152. The coil subassembly 130 may include an outer shield 136 spaced apart from the inner shield 134 and positioned adjacent and radially inward with respect to the outer lamps 152. The inner and outer shields 134, 136 may comprise a rigid material translucent to UV wavelengths, such as quartz. To help keep the spacing between the inner and outer shields 134, 136, an annular top vent plate 138 and an annular bottom vent plate 140 may be positioned between the inner and outer shields at respective top and bottom ends thereof. The top vent plate 138 may define one or more apertures 142 for providing a passageway therethrough for tubing and/or ventilation air. Similarly, the bottom vent plate 140 may define one or more apertures 144 for providing a passageway therethrough for tubing and/or ventilation air. At least one of the top or bottom vent plates 138, 140 may be removable for installing tubing 132 into and removing tubing 132 from the space between the inner and outer shields 134, 136.

The coil subassembly 130 may also include tubing 132 helically wound within the spacing between the inner and outer shields 134, 136 and having ends that may extend through the apertures 142, 144 of the top and bottom vent plates 138, 140, respectively. The tubing 132 may be Class VI tubing and comprised of a material at least partially translucent to UV light, such as FEP or PTFE or THV. The inner and outer shields 134, 136 may provide structural support for the tubing 132 and may also help insulate fluid passing through the tubing 132 from heat not directly radiated by the bulbs 154 into the fluid.

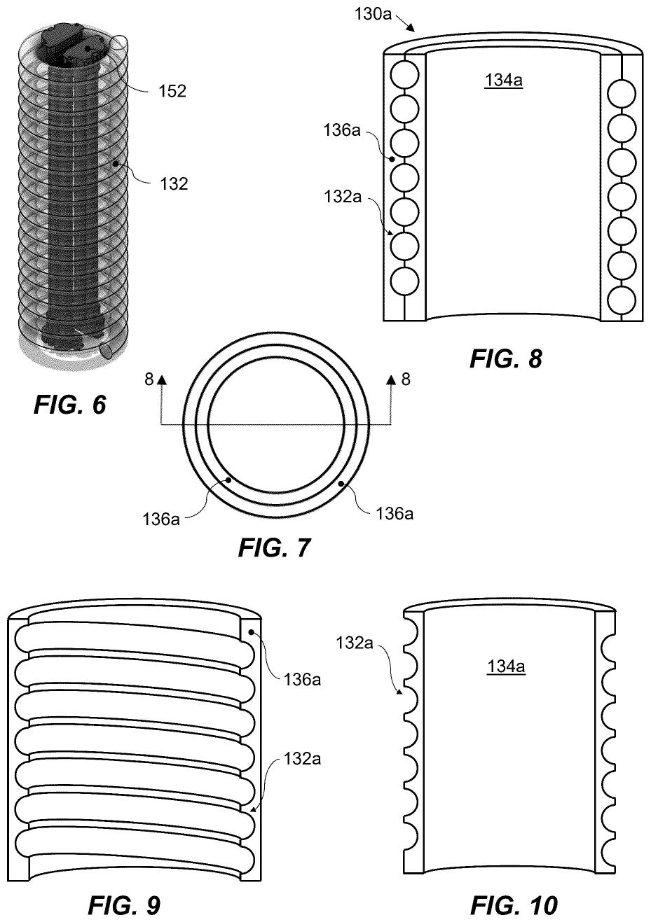

The space between the inner and outer shields 134, 136 may be configured to accommodate tubing of different diameters. For example, an operator may use a smaller diameter tubing 132, such as ¼ inch outer diameter tubing shown in FIG. 5, for a fluid that requires more extensive bombardment of photons, whereas FIG. 6 shows the inner bulbs 152 surrounded by tubing 132 having a larger diameter, such as ⅞ inch outer diameter, for a fluid that may not require as much exposure to the light emitted from the lamps 152. Thus, an operator may pass a fluid, such as the solution or a biological fluid like blood, through the photoreactor 100 with tubing 132 having a first outer diameter, such as ¼ inch. Next the operator may remove the tubing 132 from the photoreactor 100 and replace it with tubing 132 having a larger diameter, such as ⅞ inch and passing another fluid through the photoreactor 100 different from first fluid. Because the inner and outer shields 134, 136 do not contact the fluid under normal operating conditions, they are configured to remain in the photoreactor 100 during and/or after replacement of the tubing 132.

The photoreactor 100 may include a fan (not shown) housed in the space formed by the base 102 to help force air upward along the tubing 132, inner and outer shields 134, 136, and the lamp subassembly 150. In other embodiments an additional or alternative fan may be placed in the space formed by the top cap 112 to help force air out of the photoreactor 100 or downwardly through the photoreactor 100 along the aforementioned components. Alternatively, a cooling source, such as an air conditioning unit, may be configured to connect to the photoreactor 100 at the upper or lower apertures 106, 116 to force conditioned air along the aforementioned components. The photoreactor 100 may also house ballasts for the fluorescent lamps or such ballasts may be housed externally and wired to the sockets 158. It is foreseen that the sockets 158 may be configured to connected to a ballast or to bypass it for when a non-fluorescent light source is used in the photoreactor 100.

FIGS. 7-10 illustrate another exemplary coil subassembly 130a. The coil subassembly 130a includes an inner core 134a (shown in FIG. 10) defining an inner radial portion of a helically wound channel 132a and an outer cylindrical sleeve 136a (shown in FIG. 9) defining an outer radial portion of the helically wound channel 132a. The inner core 134a and the outer cylindrical sleeve 136a may comprise a material configured to at least be partially translucent to UV light, such as cyclic olefin copolymer or cyclic olefin polymer and may be formed by injection molding. The inner core 134a and the outer cylindrical sleeve 136a may be solvent bonded or ultrasonically welded to one another, as illustrated in FIG. 8, when the respective radial portions of the helically wound channel 132a are aligned. The coil subassembly 132a may be interchangeable in form and function with the coil assembly 130, including the tubing 132, inner cylindrical shield 134, outer cylindrical shield 136, top vent 138, and bottom vent 140. In some embodiments, the coil subassembly 130a may have channels 132a with a rectangular cross-section. Further, in some embodiments, the inner core 134a and outer sleeve 136a may be rectangular, which may permit the inner and/or outer lamps 152 to be arranged in linear along the coil subassembly 132a.

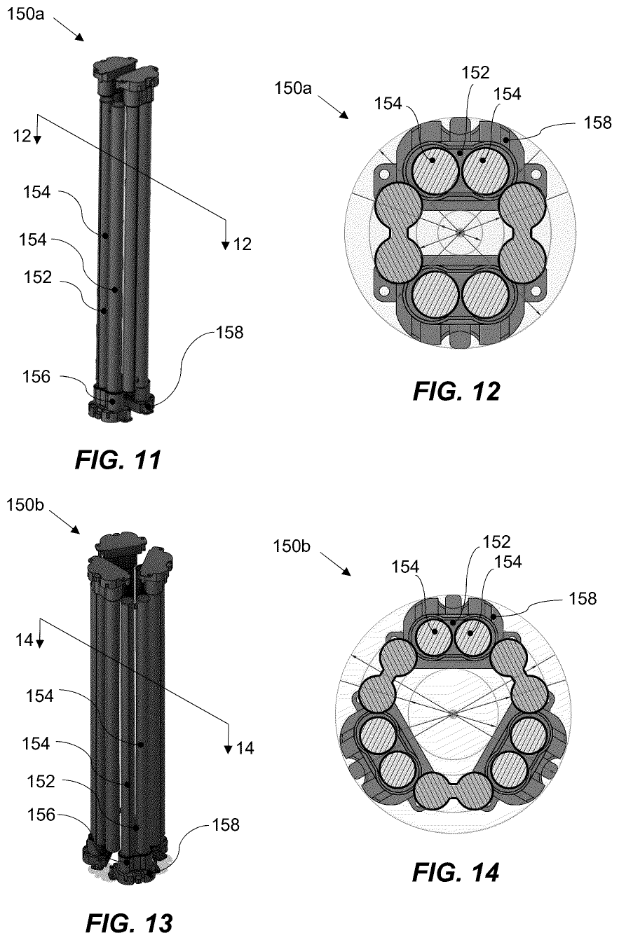

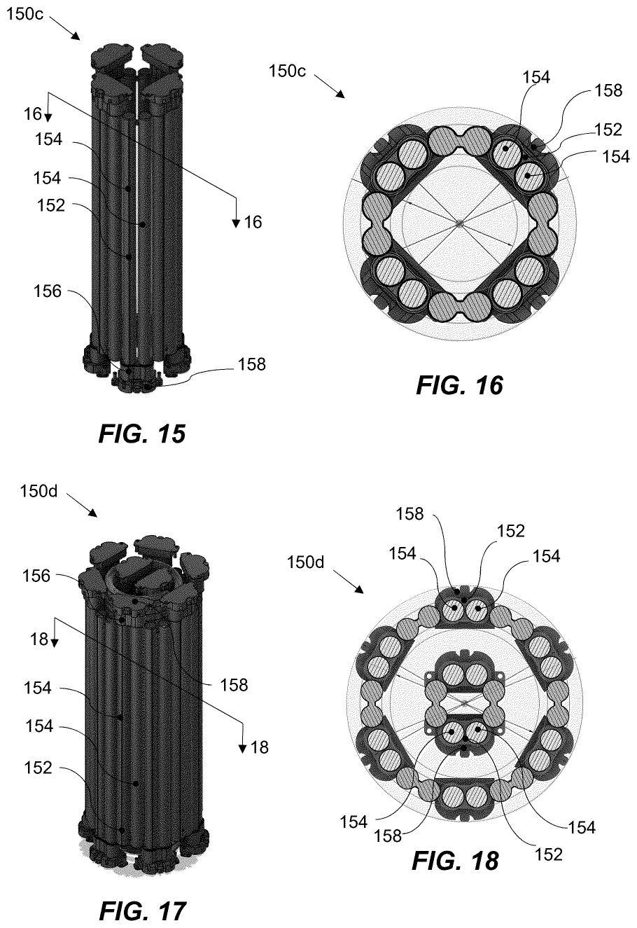

FIGS. 11 and 12 illustrate another lamp subassembly 150a similar to the lamp subassembly 150 but with only inner lamps 152. FIGS. 13 and 14 illustrate another lamp subassembly 150b similar to the lamp subassembly 150 but with six lamps 152 which may be configured as inner or outer lamps 152. FIGS. 15 and 16 illustrate another lamp subassembly 150c similar to the lamp subassembly 150 but with eight lamps 152 which may also be configured as inner or outer lamps 152. FIGS. 17 and 18 illustrate another lamp subassembly 150d similar to the lamp subassembly 150 but with six outer lamps 152. Each of lamp subassemblies 150a-150d may be interchangeable with lamp subassembly 150.

In another embodiment, the method is applied in a flow-through bioreactor such as a Couette flow device (not shown). The Couette flow device may comprise a transparent shell and LED lights surrounding the transparent shell. In some embodiments, an inner cylinder may include a thin optical shell on the outer circumference. A rotating inner cylinder may provide convection for the optical reacting layer. The inner cylinder may rotate at a sufficient speed to induce Rayleigh-Taylor vortices for efficient mixing of the mixture in the outer shell.

The inner cylinder may be suspended within an outer cylinder. The inner cylinder may be positioned between opposing ring magnets to help keep the inner cylinder centered and also to help control the axial position. The ring magnets may be radially polarized. This means that the north poles are on the outside and the south poles are on the inside, or vice versa. The rings on the stationary outer cylinder and the rotating inner cylinder may be offset axially. Either both rings on the outer cylinder are outside or inside the rings on the inner cylinder.

The inner cylinder may be constructed of thin wall aluminum. This may allow creation of eddy-currents to control the spin of the inner cylinder. Iron features may be bonded to the cylinder to create a salient-pole motor, but too much iron may create a tendency to pull the cylinder to the side wall and would have to be balanced against ring magnet force.

The size of the inner and outer cylinders may be set to provide the proper annular spacing. If the spacing is too small turbulent flow will not occur. If the spacing is too large, light penetration may be compromised.

Rotation of the inner cylinder may be controlled by a set of multiphase windings on the outer cylinder. Nominally this may be considered a three phase system. The rotating phases may drag the inner cylinder in rotation by the creation of eddy currents. A variable frequency drive should be used to allow variation of rotational speed. Light sources as discussed above may be used for illumination. Flexible OLED sheets may also be used for illumination. In addition, it may be possible to implement additional lights in the center of the stationary cylinder or on the outer surface of the rotating cylinder; these would need to be powered by inductive coupling.

The flowrate of the fluids can be controlled by the speed of the pumps. A main pump may be used to control the overall flow rate and the riboflavin pump may be slaved to the main pump to maintain the proper RF/liquid ratio.

As riboflavin is intercalated with the nucleic acids present in a fluid sample, the color of the fluid passing through the coil subassembly 130 is predictably altered. For example, as the levels of free riboflavin in solution is reduces, the color of the fluid sample to be treated changes from a strong yellow to a lighter straw-like color. Detection and measurement of this color change prior to, and after UV light treatment within the photoreactor 100 allows real-time evaluation of the photochemical reactions within fluid sample.

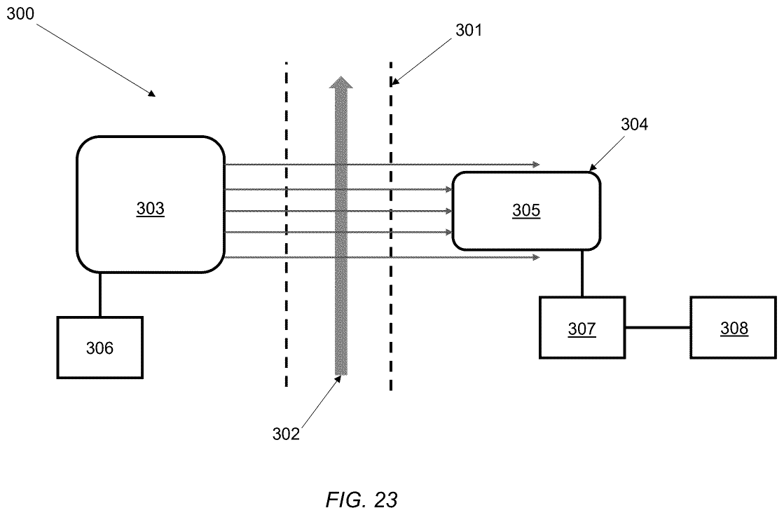

In one preferred embodiment, the photoreactor 100 of the invention includes a fluid detection assembly 300 adapted to measure the color change of the fluid sample prior to, and/or after treatment with UV light in the presence of a photosensitizer. As shown in FIG. 23, a fluid containing a quantity of riboflavin and a pathogen may pass through the coil subassembly 130 positioned between the inner and outer lamps of the lamp subassembly as described generally above. The fluid 302 of the invention may be enter and/or exit the coil subassembly 130 through a channel 301, which may be formed by tubing in fluid communication with the coil subassembly 130. Positioned adjacent to the channel 301 is a lamp 303 to emit light energy, and preferably UV light as generally described herein.

One or more photodiodes 305 are positioned opposite the lamp 303, which may be electrically connected to a power source 306, and secured by a support 304. In this embodiment, photodiodes 305 can be configured to detect light energy passing through the fluid 302 in the channel. The photodiode 305 is further electrically connected to a controller 307 that is configured to detect and amplify the captured light energy into a sensor signal that can be transmitted to an output device 308. In this embodiment, the sensor signal corresponds to the optical emission captured by the photodiode 305, which can be displayed as a visual or audible indication on an output device 308, such as general purpose computer or other computerized device configured to display sensor signal.

As noted above, in alternative embodiments, a fluid detection assembly 300 can be positioned adjacent to a fluid channel 301, preferably entering as well as exiting the coil subassembly 130 of the photoreactor 100. In this configuration, each fluid detection assembly 300 can generate a sensor signal as described. In this manner, an operator or executable computer program operating on a programable computing device, can take multiple measurements over time and monitor the changes in the sensor signals that correspond to color change in the fluid resulting from the photochemical reaction of the photosensitizer and pathogen nucleic acid in the photoreactor 100. Based on the series of sensor signal outputs, an operator or executable computer program operating on a programable computing device, can dynamically, and in real-time adjust the parameters of the photoreactor 100, such as light emission, flow rate of the fluid 302, number of illuminated lamps, concentration of photosensitizer and/or pathogen as well as wavelength of the light source.

As used herein, the “photodiode” refers to a known electronic element which comprises an electrically conducting material, in particular a semiconducting material, which exhibits a pn-junction or a PIN structure, i.e. at least two types of the material inside the photodiode, wherein the at least two types of materials comprises a different kind of doping, being denominated as “p-type” and “n-type” material, which may, further, be separated by an intrinsic “i”-type region.

As used herein, a “spectrometer,” also referred to herein as an optical spectrometer, spectrophotometer, spectrograph or spectroscope generally refers to an instrument used to measure properties of light over a specific portion of the electromagnetic spectrum, typically used in spectroscopic analysis

As used herein, the term “light” generally refers to electromagnetic radiation in one or more of the visible spectral range, the ultraviolet spectral range and the infrared spectral range. Therein, the term visible spectral range generally refers to a spectral range of 380 nm to 780 nm. The term infrared spectral range generally refers to electromagnetic radiation in the range of 780 nm to 1 mm, preferably in the range of 780 nm to 3.0 micrometers. The term ultraviolet spectral range generally refers to electromagnetic radiation in the range of 1 nm to 380 nm, preferably in the range of 100 nm to 380 nm. Preferably, light as used within the present invention is visible light, i.e. light in the visible spectral range. The term light beam generally refers to an amount of light emitted and/or reflected into a specific direction. Thus, the light beam may be a bundle of the light rays having a predetermined extension in a direction perpendicular to a direction of propagation of the light beam. Preferably, the light beams may be or may comprise one or more Gaussian light beams which may be characterized by one or more Gaussian beam parameters, such as one or more of a beam waist, a Rayleigh-length or any other beam parameter or combination of beam parameters suited to characterize a development of a beam diameter and/or a beam propagation in space.

As used herein, “photosensitizer” generally refers to a chemical compound that absorbs electromagnetic radiation, most commonly in the visible spectrum, and releases it as another form of energy, most commonly as reactive oxygen species and/or as thermal energy. Preferably, the compound is nontoxic to humans or is capable of being formulated in a nontoxic composition. Preferably, the chemical compound in its photodegraded form is also nontoxic. A non-exhaustive list of photosensitive chemicals may be found in Kreimer-Birnbaum, Ser. Hematol. 26:157-73, 1989 and in Redmond and Gamlin, Photochem. Photbiol. 70 (4): 391-475 (1999) both of which are incorporated herein by reference. examples of photosensitizers can include flavins, such as riboflavin or psoralen.

As used herein, “flavin” generally refers to a group of organic compounds based on pteridine, formed by the tricyclic heteronuclear organic ring isoalloxazine and derivatives thereof, such as for example riboflavin:

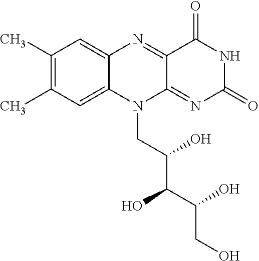 [SMILES: Cc1cc2nc3c(=O)[nH]c(=O)nc-3n(C[C@H](O)[C@H](O)[C@H](O)CO)c2cc1C]

As used herein, a “psoralen” generally refers to a photo-reactive parent compound in a family of naturally occurring organic compounds known as the linear furanocoumarins. Psoralen “psoralen” refers to a natural compound which forms DNA interstrand cross-links by intercalating into DNA at 5′-AT sequences, wherein the psoralen binds and forms thymidine adducts with the thymidine nucleotide in the presence of UVA irradiation, in one embodiment, a psoralen includes a compound having the following chemical formula:

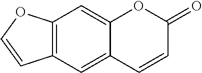 [SMILES: O=c1ccc2cc3ccoc3cc2o1]

As used herein, a “sensor signal” generally refers to an arbitrary memorable and transferable signal which is generated by a photodiode in response to illumination. Thus, as an example, the sensor signal may be or may comprise at least one electronic signal, which may be or may comprise a digital electronic signal and/or an analogue electronic signal. The sensor signal may be or may comprise at least one voltage signal and/or at least one current signal. Further, either raw sensor signals may be used, or the detector, the optical sensor or any other element may be adapted to process or preprocess the sensor signal, thereby generating secondary sensor signals, which may also be used as sensor signals, such as preprocessing by filtering or the like. The sensor signal may generally be an arbitrary signal indicative of light intensity, and preferably light intensity over time.

Certain embodiments of the inventive technology may utilize a machine and/or device, such as a module, which may include a general purpose computer, a computer that can perform an algorithm, computer readable medium, software, computer readable medium continuing specific programming, a computer network, a server and receiver network, transmission elements, wireless devices and/or smart phones, internet transmission and receiving element; cloud-based storage and transmission systems, software updatable elements; computer routines and/or subroutines, computer readable memory, data storage elements, random access memory elements, and/or computer interface displays that may represent the data in a physically perceivable transformation such as visually displaying said processed data. In addition, as can be naturally appreciated, any of the steps as herein described may be accomplished in some embodiments through a variety of hardware applications including a keyboard, mouse, computer graphical interface, voice activation or input, server, receiver and any other appropriate hardware device known by those of ordinary skill in the art.

As used herein a “controller” may include a “processor,” “processor system,” or “processing system,” which includes any suitable hardware and/or software system, mechanism or component that processes data, sensor signals or other information. A processor can include a system with a general-purpose central processing unit, multiple processing units, dedicated circuitry for achieving functionality, or other systems and for implementing one or more “computer executable program,” generally in the form of programed software-based executable instructions. Processing need not be limited to a geographic location or have temporal limitations. For example, a processor can perform its functions in “real time,” “offline,” in a “batch mode,” etc. Portions of processing can be performed at different times and at different locations, by different (or the same) processing systems. A computer may be any processor in communication with a memory. The memory may be any suitable processor-readable storage medium, such as random-access memory (RAM), read-only memory (ROM), magnetic or optical disk, or other tangible media suitable for storing instructions for execution by the processor.

Particular embodiments may be implemented by using a programmed general purpose digital computer, by using application specific integrated circuits, programmable logic devices, field programmable gate arrays, optical, chemical, biological, quantum or nano-engineered systems, components and mechanisms may be used. In general, the functions of particular embodiments can be achieved by any means as is known in the art. Distributed, networked systems, components, and/or circuits can be used. Communication, or transfer, of data may be wired, wireless, or by any other means.

It will also be appreciated that one or more of the elements depicted in the drawings/figures can also be implemented in a more separated or integrated manner, or even removed or rendered as inoperable in certain cases, as is useful in accordance with a particular application. It is also within the spirit and scope to implement a program or code that can be stored in a machine-readable medium to permit a computer to perform any of the methods described above.

For the sake of brevity, conventional techniques related to computer programming, computer networking, and other functional aspects of the systems (and the individual operating components of the systems) may not be described in detail herein. In addition, those skilled in the art will appreciate that embodiments may be practiced in conjunction with any number of system and/or network architectures, data transmission protocols, and device configurations, and that the system described herein is merely one suitable example. Furthermore, certain terminology may be used herein for the purpose of reference only, and thus is not intended to be limiting. For example, the terms “first”, “second” and other such numerical terms do not imply a sequence or order unless clearly indicated by the context.

Embodiments of the subject matter may be described herein in terms of functional and/or logical block components and with reference to symbolic representations of operations, processing tasks, and functions that may be performed by various computing components or devices. Such operations, tasks, and functions are sometimes referred to as being computer-executed, computerized, software-implemented, or computer-implemented. In this regard, it should be appreciated that the various block components shown in the figures may be realized by any number of hardware, software, and/or firmware components configured to perform the specified functions.

For example, an embodiment of a system or a component may employ various integrated circuit components, e.g., memory elements, digital signal processing elements, logic elements, look-up tables, or the like, which may carry out a variety of functions under the control of one or more microprocessors or other control devices. In this regard, the subject matter described herein can be implemented in the context of any computer-implemented system and/or in connection with two or more separate and distinct computer-implemented systems that cooperate and communicate with one another.

As used herein, the term “electrical connected” means two or more components of a system that are configured to allow the wired or wireless transmission of an electrical current.

Further, if or when used, the use of the transitional phrase “comprising” is used to maintain the “open-end” claims herein, according to traditional claim interpretation. Thus, unless the context requires otherwise, it should be understood that the term “comprise” or variations such as “comprises” or “comprising”, are intended to imply the inclusion of a stated element or step or group of elements or steps but not the exclusion of any other element or step or group of elements 5 or steps. Such terms should be interpreted in their most expansive form so as to afford the applicant the broadest coverage legally permissible.

It should be understood from the foregoing that, while particular aspects have been illustrated and described, various modifications can be made thereto without departing from the spirit and scope of the invention as will be apparent to those skilled in the art. Such changes and modifications are within the scope and teachings of this invention as defined in the claims appended hereto.

1. A system for detecting light emitted into a photoreactor comprising:
a housing containing:
a fluid channel configured to transfer a fluid containing a photosensitizer and a reactant from an inlet to an outlet along a longitudinal axis; and
at least one light source positioned adjacent to the fluid channel along the longitudinal axis;
a light detection assembly comprising:
one or more photodiodes positioned adjacent to the light source and configured to generate an electrical signal in response to the light energy produced by the at least one light source;
a controller electrically connected to said one or more photodiodes, configured to convert said electrical signal to a sensor signal that corresponds to said light energy.

2-20. (canceled)

21. A system for measuring a photochemical reaction comprising:
a photoreactor having a fluid channel configured to transfer a fluid from an inlet to an outlet, wherein said fluid comprises a photosensitizer and a reactant;
a fluid detection assembly positioned at the inlet and/or outlet of said fluid channel comprising:
a light source configured to direct light energy through said fluid in the fluid channel;
at least one photodiode positioned adjacent to said fluid channel and configured to generate an electrical signal in response to the light energy produced by the light source;
a controller, electrically connected to said one or more photodiodes, configured to convert said electrical signal to a sensor signal that corresponds to said light energy passing through the fluid.

22. The system of claim 21, wherein said photosensitizer is selected from: a flavin, a psoralen, and riboflavin.

23. (canceled)

24. The system of claim 22, wherein said reactant comprises a microorganism selected from: a virus, and/or a bacterium.

25-26. (canceled)

27. The system of claim 21, wherein said sensor signal corresponds to a color change in the fluid.

28. The system of claim 21, wherein said photodiode comprises a UV-A dominant diode.

29. The system of claim 28, wherein said UV-A dominant diode has a detection sensitivity between 220 nm-370 nm.

30. The system of claim 29, wherein the intensity of the light source is adjusted in response to the sensor signal, or wherein one or more light sources are activated or deactivated in response to the sensor signal.

31. (canceled)

32. The system of claim 21, wherein in response to the sensor signal one or more of the following occur:
the wavelength of the light source is adjusted;
the ratio of the photosensitizer and the reactant is adjusted in response to the sensor signal;
the controller automatically adjusts in response to the sensor signal; or
controller automatically adjusts in response to the sensor signal in real-time.

33-35. (canceled)

36. The system of claim 21, wherein said light source is selected from: an LED light source, a narrowband wavelength light source, a fluorescent light source, and a peak UV-B wavelength, a peak UV-C wavelength, and a peak wavelength outside of UV-B and UV-C.

37-77. (canceled)

78. A system for detecting emitted light comprising:
a fluid channel configured to transfer a fluid containing at least one reactant; and
at least one light source positioned adjacent to the fluid channel;
a light detection assembly comprising:
one or more sensors positioned adjacent to the light source and configured to detect the light energy produced by the light source;
a controller electrically connected to said one or more sensors, configured to generate a signal that corresponds to said wavelength of the light energy or the color spectra of the fluid.

79. The system of claim 78, wherein the one or more sensors comprises one or more photodiodes.

80. The system of claim 79, wherein the one or more photodiodes are positioned adjacent to the light source and configured to generate an electrical signal in response to the wavelength of the light energy produced by the at least one light source.

81. The system of claim 78, wherein said reactant is selected from: a chemical reactant, a buffer, a photo reactant, microorganism.

82-84. (canceled)

85. The system of claim 81, wherein said microorganism is selected from: a virus, and/or a bacterium.

86. The system of claim 78, wherein the intensity of the light source is adjusted in response to the signal, or wherein one or more light sources are activated or deactivated in response to the signal.

87. (canceled)

88. The system of claim 78, wherein in response to the signal one or more of the following occurs:
the wavelength of the light source is adjusted in response to the signal;
the ratio of the fluid and the reactant is adjusted in response to the signal; or
the flow rate of the fluid through the channel is adjusted in response to the signal.

89-90. (canceled)

91. The system of claim 78, wherein the light source is a selected from: a fluorescent light source, an LED light source, a narrowband wavelength light source, a peak UV-B wavelength, a peak UV-C wavelength, and a peak wavelength outside of UV-B and UV-C.

92-119. (canceled)
